(19) United States
(12) Patent Application Publication
(10) Pub. No.: US 2024/0009428 A1
(43) Pub. Date: Jan. 11, 2024

(54) CATHETER ASSEMBLY PLUG DEVICES, SYSTEMS, AND METHODS

(71) Applicant: Becton, Dickinson and Company, Franklin Lakes, NJ (US)

(72) Inventors: Sankaranarayanan Thirumoorthy, Tamil Nadu (IN); Mukilan Balamurugan, Tamil Nadu (IN); Rajdeep Das Pradeep, Sandy, UT (US); Darin Vincent, Salt Lake City, UT (US); Sivaramakrishnan Balasubramanian, Cary, NC (US)

(21) Appl. No.: 18/033,772

(22) PCT Filed: Oct. 27, 2021
(86) PCT No.: PCT/US2021/056778

§ 371 Date: Apr. 25, 2023

(30) Foreign Application Priority Data
Nov. 12, 2020 (IN) 202011049485

(51) Int. Cl. A61M 25/06

(52) U.S. Cl. CPC A61M 25/0693

(57) ABSTRACT

A plug may be used to plug a proximal end of a flashback chamber or an adapter in an intravenous catheter assembly. The plug may provide air venting of the flashback chamber or the adapter. The plug may include a body and a neck portion extending from the body. The neck portion may include one or more air vent channels, which may be aligned with a longitudinal axis of the plug. The neck portion may include one or more transverse channels, which may provide air venting and/or blood collection in case of leakage of blood into the plug.

### BACKGROUND

Intravenous catheters are commonly used for a variety of infusion therapies. For example, intravenous catheters may be used for infusing fluids, such as normal saline solution, various medicaments, and total parenteral nutrition, into a patient. Intravenous catheters may also be used for withdrawing blood from the patient.

Common types of intravenous catheter are peripheral IV catheters (“PIVCs”), peripherally inserted central catheters (“PICCs”), and midline catheters. Intravenous catheters may include “over-the needle” catheters, which may be mounted over a needle having a sharp distal tip. The sharp distal tip may be used to pierce skin and the vasculature of the patient. Insertion of the intravenous catheter into the vasculature may follow the piercing of the vasculature by the needle. The needle and the intravenous catheter are generally inserted at a shallow angle through the skin into the vasculature of the patient with a bevel of the needle facing up and away from the skin of the patient.

In order to verify proper placement of the introducer needle and/or the intravenous catheter in the vasculature, a user generally confirms that there is flashback of blood, which may be visible to the user. In some instances, the introducer needle may include a notch disposed towards a distal end of the introducer needle, and in response to the distal tip of the introducer needle being positioned within the vasculature, blood may flow proximally through a needle lumen, exit the needle lumen through the notch, and then travel proximally between an outer surface of the introducer needle and an inner surface of the intravenous catheter.

Accordingly, where the intravenous catheter is at least partially transparent, the user may visualize a small amount of blood “flashback” and thereby confirm placement of the intravenous catheter within the vasculature. Presence of a vasculature entrance indicator, such as flashback, may facilitate successful placement of intravenous catheters. Once placement of the introducer needle within the vasculature has been confirmed, the user may temporarily occlude flow in the vasculature and withdraw the introducer needle, leaving the intravenous catheter in place for future blood withdrawal and/or fluid infusion.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure generally relates to plug devices, systems, and methods that facilitate venting. In some embodiments, a plug may be used to plug a proximal end of a flashback chamber in an intravenous catheter assembly. In some embodiments, the plug may provide air venting of the flashback chamber. In some embodiments, the plug may include a body, which may include a cavity and a vent hole extending distally from the cavity.

In some embodiments, the plug may include a neck portion, which may extend from a distal end of the body. In some embodiments, the neck portion may include one or more annular grooves spaced apart by one or more disc-shaped portions. In some embodiments, a proximal-most disc-shaped portion of the disc-shaped portions may be disposed proximate the body. In some embodiments, an outer edge of each of the disc-shaped portions may include an air vent channel, which may be generally aligned with a longitudinal axis of the plug. In some embodiments, the air vent channel of the proximal-most disc-shaped portion may be aligned with the vent hole.

In some embodiments, a distal-most one of disc-shaped portion of the disc-shaped portions may form a distal end of the neck portion. In some embodiments, the proximal-most disc-shaped portion and the distal end of the body may form a stepped surface. In some embodiments, a proximal end of the body may include an opening of the cavity. In some embodiments, a surface of the cavity may include multiple of annular protrusions.

In some embodiments, the neck portion may include one or more holes extending through the neck portion. In some embodiments, the holes may be generally perpendicular to a longitudinal axis of the plug. In some embodiments, an air pathway through the plug may include the holes, the air vent channels, the vent hole, and the cavity. In some embodiments, the holes may be spaced apart in a distal-proximal direction along the neck portion.

In some embodiments, a first hole of the holes may be adjacent to a second hole of the holes. In some embodiments, the second hole may be adjacent to a third hole of the holes. In some embodiments, a first air vent channel may extend between the first hole and the second hole. In some embodiments, a second air vent channel may extend between the second hole and the third hole. In some embodiments, the first air vent channel may be disposed on an opposite side of the neck portion as the second air vent channel.

In some embodiments, the third hole may be adjacent to a fourth hole of the holes. In some embodiments, a third air vent channel may extend between the third hole and the fourth hole. In some embodiments, the third air vent channel may be disposed on a same side of the neck portion as the first air vent channel. In some embodiments, the holes may include a proximal-most hole. In some embodiments, the vent hole may intersect the proximal-most hole. In some embodiments, a proximal end of the neck portion and the distal end of the body may form a stepped surface.

In some embodiments, the plug may be used for plugging a port of an adapter in the intravenous catheter assembly. In some embodiments, the plug may provide air venting of the port. In some embodiments, the neck portion may extend from the body. In some embodiments, one or more longitudinal air vent channels may be generally aligned with a longitudinal axis of the plug. In some embodiments, one or more transverse air vent channels may connect the longitudinal air vent channels to provide an air pathway from a distal end of the neck portion.

In some embodiments, the longitudinal air vent channels and the transverse longitudinal air vent channels may pass air but not blood. In some embodiments, the body may include an end wall and a skirt extending from the end wall to define an annular receiving channel between the skirt and the neck portion. In some embodiments, an inner surface of the skirt may include threading. In some embodiments, the end wall may include one or more vent holes extending there through. In some embodiments, an inner surface of the skirt may include multiple snap features.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example catheter assembly, according to some embodiments;

FIG. 1B is an upper perspective view of an example plug, according to some embodiments;

FIG. 1C is a cross-sectional view of a portion of the catheter assembly of FIG. 1A, according to some embodiments;

FIG. 1D is an enlarged cross-sectional view of a portion of the catheter assembly of FIG. 1A, according to some embodiments;

FIG. 1E is an enlarged upper perspective view of a portion of the plug of FIG. 1B, illustrating multiple example air vent channels and a portion of an example annular groove, according to some embodiments;

FIG. 1F is a cross-sectional view of the catheter assembly of FIG. 1A along the line 1F-1F of FIG. 1A, according to some embodiments;

FIG. 1G is a cross-sectional view of the catheter assembly of FIG. 1A along the line 1G-1G of FIG. 1A, according to some embodiments;

FIG. 1H is a cross-sectional view of the catheter assembly of FIG. 1A along the line 1H-1H of FIG. 1A, according to some embodiments;

FIG. 2A is an upper perspective view of the catheter assembly, according to some embodiments;

FIG. 2B is an upper perspective view of another example plug, according to some embodiments;

FIG. 2C is a cross-sectional view of a portion of the catheter assembly of FIG. 2A, according to some embodiments;

FIG. 2D is an enlarged cross-sectional view of a portion of the catheter assembly of FIG. 2A, according to some embodiments;

FIG. 2E is a cross-sectional view of the catheter assembly of FIG. 2A along the line 2E-2E of FIG. 2A, according to some embodiments;

FIG. 2F is a cross-sectional view of the catheter assembly of FIG. 2A along the line 2F-2F of FIG. 2A, according to some embodiments;

FIG. 2G is a cross-sectional view of the catheter assembly of FIG. 2A along the line 2G-2G of FIG. 2A, according to some embodiments;

FIG. 3A is an upper perspective view of another example plug, according to some embodiments;

FIG. 3B is an upper perspective view of the plug of FIG. 3A, according to some embodiments;

FIG. 3C is an upper perspective view of the plug of FIG. 3A, according to some embodiments;

FIG. 3D is an upper perspective view of the plug of FIG. 3A, according to some embodiments;

FIG. 3E is an upper perspective view of the plug of FIG. 3A, according to some embodiments;

FIG. 3F is an upper perspective view of the plug of FIG. 3A, according to some embodiments;

FIG. 3G is an upper perspective view of the plug of FIG. 3A, according to some embodiments;

FIG. 3H is an upper perspective view of the plug of FIG. 3A, according to some embodiments;

FIG. 3I is a graph of hydraulic resistance;

FIG. 4A is an upper perspective view of another example plug that includes a Luer slip connector, according to some embodiments;

FIG. 4B is an upper perspective view of the plug of FIG. 4A, illustrating an example proximal end of the plug, according to some embodiments;

FIG. 4C is a cross-sectional view of an example Y-adapter coupled to the plug of FIG. 4A, according to some embodiments;

FIG. 5A is an upper perspective view of another example plug that includes a Luer thread connector, according to some embodiments;

FIG. 5B is an upper perspective view of the Y-adapter of FIG. 4B coupled to the plug of FIG. 5A, according to some embodiments;

FIG. 5C is a cross-sectional view of a portion of the Y-adapter and the plug of FIG. 5A, according to some embodiments;

FIG. 6A is an upper perspective view of another example plug that includes a Luer snap connector, according to some embodiments;

FIG. 6B is an upper perspective view of the Y-adapter of FIG. 4B coupled to the plug of FIG. 6A, according to some embodiments;

FIG. 6C is a cross-sectional view of a portion of the Y-adapter and the plug of FIG. 6A, according to some embodiments;

FIG. 7 is an upper perspective view of another catheter assembly, according to some embodiments;

FIG. 8A is a cross-sectional view of another catheter assembly, illustrating another example plug, according to some embodiments;

FIG. 8B is an upper perspective view of the plug of FIG. 8A, according to some embodiments;

FIG. 8C is a lower perspective view of the plug of FIG. 8A, according to some embodiments;

FIG. 8D is an upper perspective view of the plug of FIG. 8A, according to some embodiments;

FIG. 8E is a lower perspective view of the plug of FIG. 8A, according to some embodiments;

FIG. 9A is a cross-sectional view of a portion of the Y-adapter of FIG. 4B coupled to another plug, illustrating the plug in a first position, according to some embodiments;

FIG. 9B is a cross-sectional view of the portion of the Y-adapter and plug of FIG. 9A, illustrating the plug in a second or venting position, according to some embodiments; and

FIG. 9C is an upper perspective view of the Y-adapter and plug of FIG. 9A, according to some embodiments.

### DESCRIPTION OF EMBODIMENTS

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

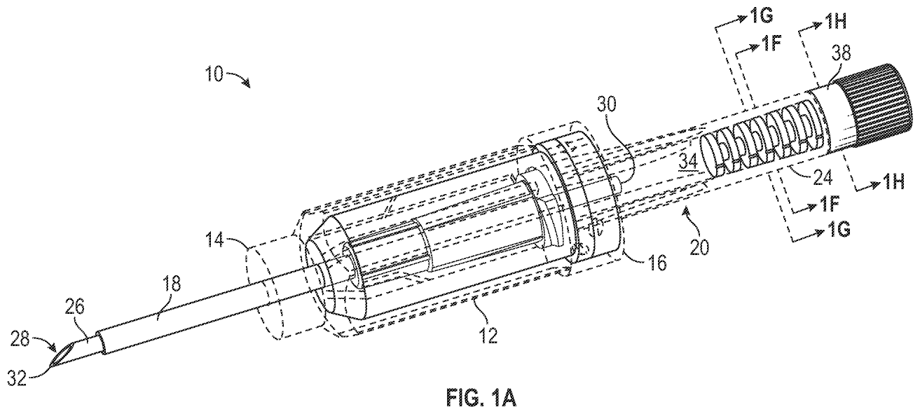

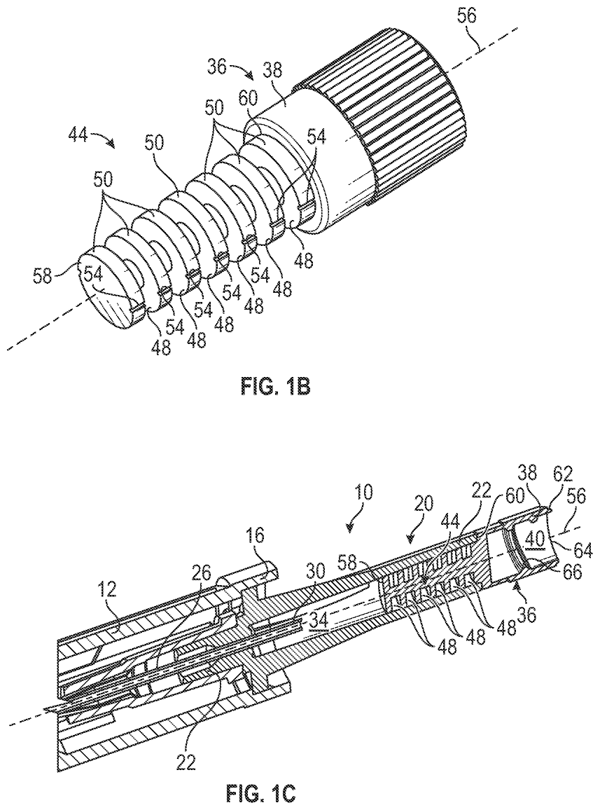

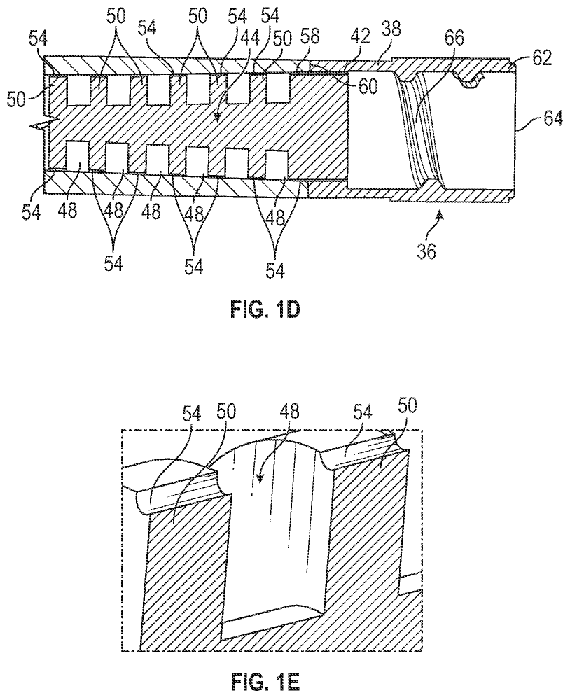

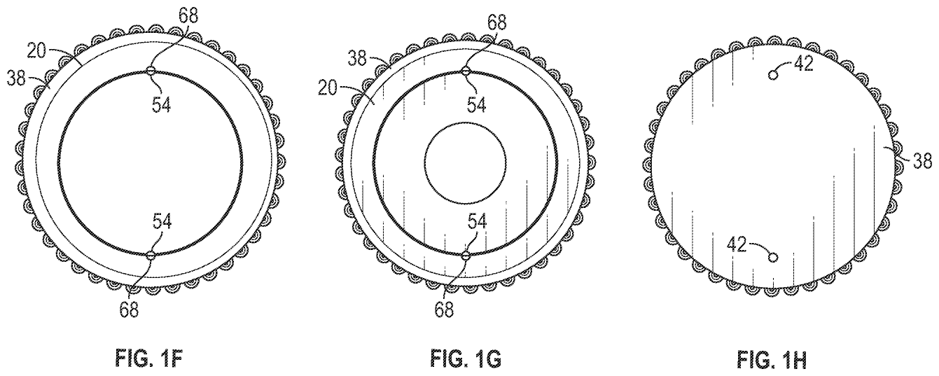

Referring now to FIG. 1A, a catheter assembly 10 is illustrated, according to some embodiments. In some embodiments, the catheter assembly 10 may include a catheter adapter 12 which may include a distal end 14, a proximal end 16, and a lumen extending through the distal end 14 and the proximal end 16. In some embodiments, the catheter assembly 10 may include a catheter 18 extending distally from the distal end 14 of the catheter adapter 12. In some embodiments, the catheter assembly 10 may include a needle hub 20, which may include a distal end 22 and a proximal end 24. In some embodiments, the distal end 22 of the needle hub 20 may be coupled to the proximal end 16 of the catheter adapter 12.

In some embodiments, the catheter assembly 10 may include a peripheral intravenous catheter assembly, such as, for example, the BD NEXIVA™ Closed IV Catheter System, the BD NEXIVA™ DIFFUSICS™ Closed IV Catheter System, the BD SAF-T-INTIMA™ Closed IV Catheter System, the BD INSYTE™ AUTOGUARD™ BC Shielded Catheter, the BD ANGIOCATH™ AUTOGUARD™ Shielded IV Catheter, or another suitable intravenous catheter assembly. In some embodiments, the catheter assembly 10 may include a peripheral inserted central catheter (“PICC”) assembly or a midline catheter assembly.

In some embodiments, the catheter assembly 10 may include a needle 26, which may include a distal end 28, a proximal end 30, and a lumen extending there between. In some embodiments, the needle 26 may include an introducer needle, which may include a sharp distal tip 32. In some embodiments, the needle 26 may be secured within the needle hub 20. In some embodiments, the needle 26 may be constructed of metal or another suitable material.

In some embodiments, the needle hub 20 may include a flashback chamber 34. In some embodiments, in response to the sharp distal tip 32 being positioned within vasculature of a patient, blood may flow proximally through the lumen of the needle 26 and exit the proximal end 30 of the needle 26 into the flashback chamber 34. In some embodiments, the needle hub 20 may be transparent, which may facilitate observation of blood within the flashback chamber 34 by a user. Accordingly, in some embodiments, the user may visualize a small amount of blood “flashback” and thereby confirm placement of the catheter 18 within the vasculature.

In some embodiments, the catheter assembly 10 may include a plug 36, which may be coupled to the proximal end 24 of the needle hub 20. In some embodiments, the plug 36 may be used to plug or seal a proximal end of the flashback chamber 34 in the catheter assembly 10. In some embodiments, the plug 36 may provide air venting of the flashback chamber 34. In some embodiments, the plug 36 may prevent or reduce leakage of blood from the proximal end 16 of the needle hub 20, as will be explained in further detail.

Referring now to FIGS. 1B-1H, in some embodiments, the plug 36 may include a body 38, which may include a cavity 40. In some embodiments, the body 38 may include one or more vent holes 42 that may extend distally from the cavity 40. In some embodiments, the plug 36 may include a neck portion 44, which may extend from a distal end 46 of the body 38. In some embodiments, the neck portion 44 may include one or more annular grooves 48 spaced apart by one or more disc-shaped portions 50. In some embodiments, the annular grooves 48 may correspond to transverse channels.

In some embodiments, a most-proximal of the disc-shaped portions 50 may be disposed proximate the body 38. In some embodiments, an outer edge of each of the disc-shaped portions may include one or more air vent channels 54, which may be generally aligned with a longitudinal axis 56 of the plug 36. In some embodiments, the air vent channels 54 of the proximal-most one of the disc-shaped portions 50 may be aligned with and/or proximate the vent holes 42.

In some embodiments, an air pathway through the plug 36 may include the annular grooves 48, the air vent channels 54, the vent holes 42, and the cavity 40. In further detail, in some embodiments, in response to flashback entering the flashback chamber 34, air may flow proximally through the air vent channels 54 and the annular grooves 48 until it reaches the vent holes 42. In some embodiments, the air may then flow proximally through the vent holes 42 into the cavity 40, which may contact an external environment.

In some embodiments, the air vent channels 54 may be configured to allow air but not blood to pass. However, in other embodiments, blood may leak through the air vent channels 54 and through the air pathway, such that the air pathway is also a fluid pathway that includes blood. In some embodiments, the plug 36 may incorporate sudden contraction, sudden expansion, and bending loss, which may control and reduce blood flow and leakage. In some embodiments, the annular grooves 48 may provide sudden expansion and bending loss, while the air vent channels 54 may provide sudden contraction. In some embodiments, the annular grooves 48 and the air vent channels 54 may delay blood leakage through the plug 36. In some embodiments, the annular grooves 48 may hold a substantial blood volume, which may delay blood leakage while allowing the plug 36 to be relatively short. In some embodiments, the annular grooves 48 may be deeper than the air vent channels 54.

In some embodiments, unlike vent plugs known in the art, the plug 36 may not include a membrane to delay blood leakage due to the presence of the annular grooves 48 and the air vent channels 54. In some embodiments, the plug 36 may include a membrane in addition to the annular grooves 48 and/or the air vent channels 54. In some embodiments, the neck portion 44 may include a first line of the air vent channels 54 opposite a second line of the air vent channels 54, as illustrated, for example, in FIG. 1D, which may slow blood leakage. In some embodiments, the neck portion 44 may include various other patterns of the air vent channels 54.

In some embodiments, a distal-most one of the disc-shaped portions 50 may form the distal end 58 of the neck portion 44. In some embodiments, the disc-shaped portions 50 may span a lumen of the needle hub 20, and the outer edge of each of the disc-shaped portions 50 may contact an inner wall of the needle hub 20. In some embodiments, the outer edge of each of the disc-shaped portions 50 may be generally circular. In some embodiments, the neck portion 44 may be tapered to match a shape of the lumen of the needle hub 20 and securely fit within the needle hub 20. In some embodiments, the proximal-most one of the disc-shaped portions 50 and the distal end 46 of the body 38 may form a stepped surface 60, which may be annular. In some embodiments, the stepped surface 60 may abut the proximal end 16 of the needle hub 20.

In some embodiments, a proximal end 62 of the body 38 may include an opening 64 of the cavity 40. In some embodiments, the plug 36 may prevent or reduce leakage of blood outside or proximal to the needle hub 20. For example, a surface of the cavity 40 may include one or more annular protrusions 66, which may delay blood leakage within the body 38. As illustrated in FIGS. 1F-1G, in some embodiments, an inner surface of the needle hub 20 may include one or more channels 68 that align with the air vent channels 54.

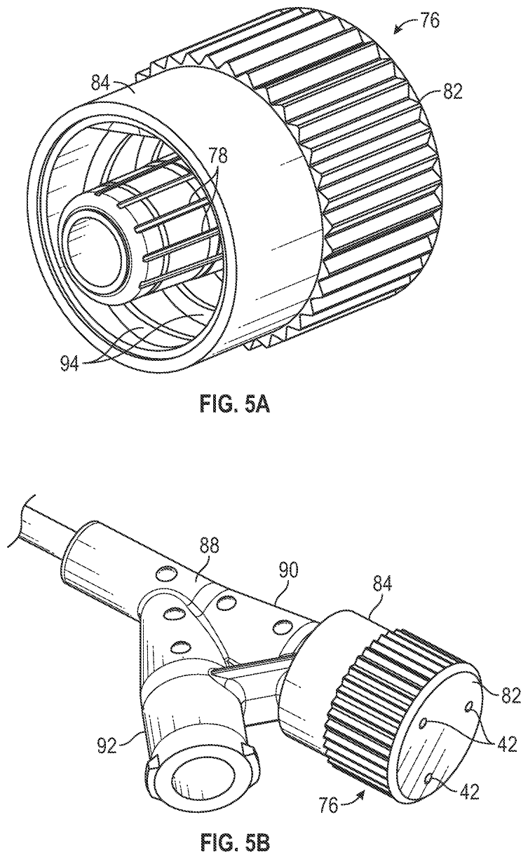

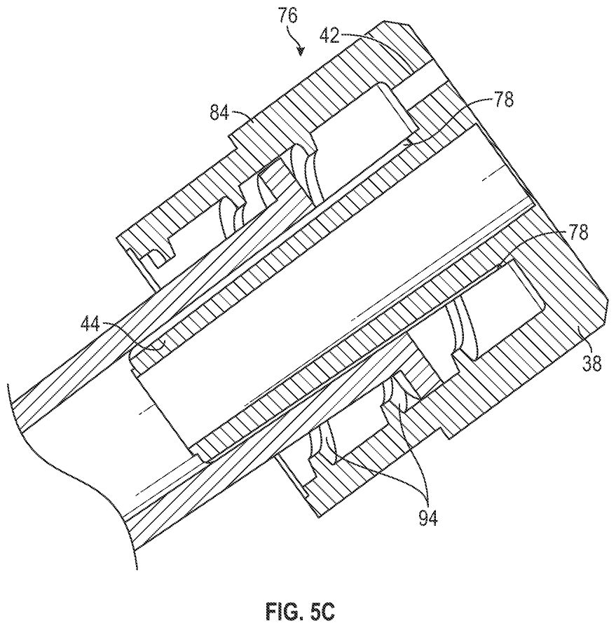

In some embodiments, the plug 36 may be used to plug a port of an adapter of a particular catheter assembly, such as, for example, the adapter described with respect to FIG. 5B. In some embodiments, the plug 36 may be coupled to the port of the adapter and may provide air venting of the adapter. In some embodiments, the plug 36 may prevent or reduce leakage of blood from the port. In some embodiments, the disc-shaped portions 50 may span a lumen of the adapter, and the outer edge of each of the disc-shaped portions 50 may contact an inner wall of the adapter. In some embodiments, the stepped surface 60 may abut the port of the adapter.

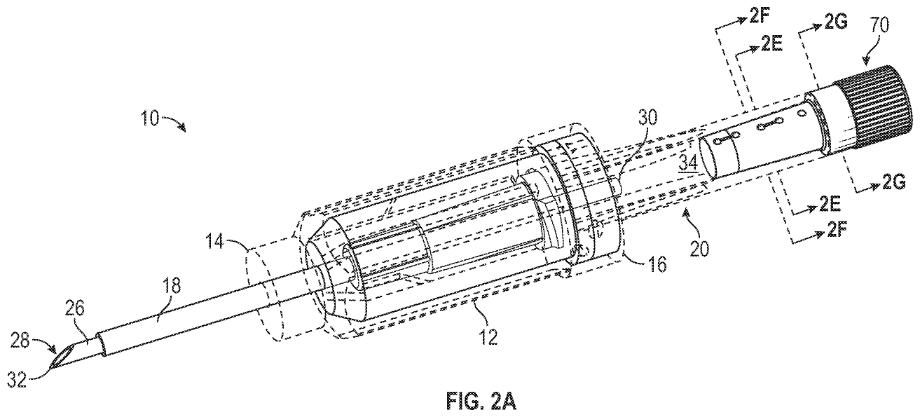

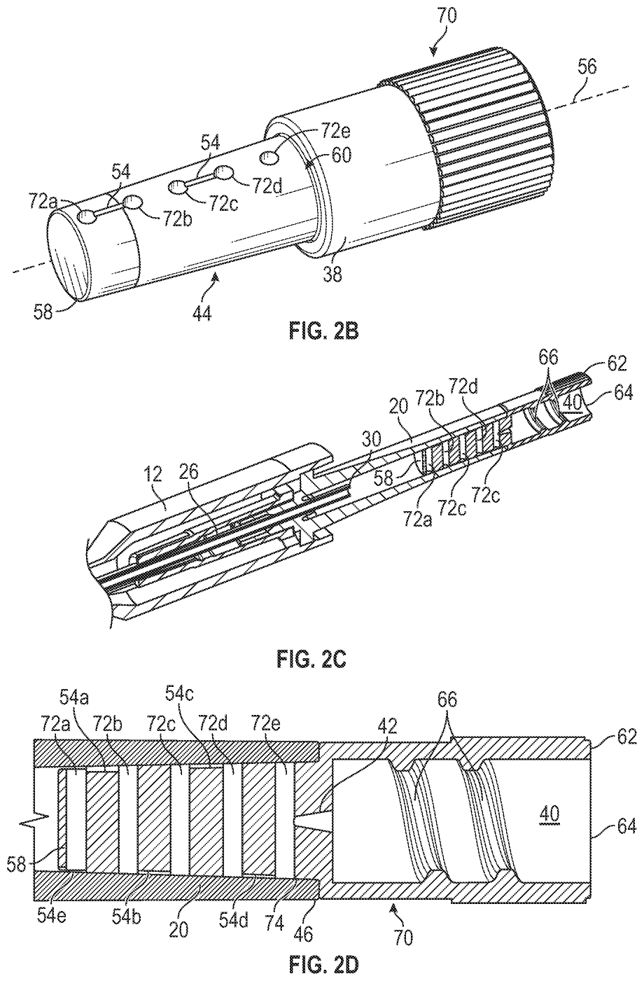

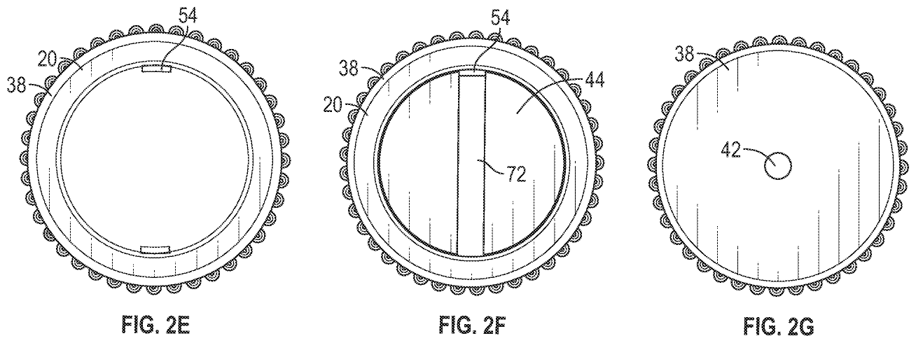

Referring now to FIGS. 2A-2G, the catheter assembly 10 is illustrated, according to some embodiments. In some embodiments, the catheter assembly 10 may include a plug 70. In some embodiments, the plug 70 may be similar or identical to the plug 36 described with respect to FIGS. 1A-1H in terms of one or more included components and/or operation.

In some embodiments, the neck portion 44 of the plug 70 may include one or more holes 72 extending through the neck portion 44. In some embodiments, the holes 72 may be generally perpendicular to the longitudinal axis 56 of the plug 70. In some embodiments, the holes 72 may be spaced apart in a distal-proximal direction along the neck portion 44, as illustrated, for example, in FIG. 2B. In some embodiments, the holes 72 may be axially aligned. In some embodiments, the neck portion 44 may include a truncated cone or a cylinder, which may allow the neck portion 44 to snugly fit within the needle hub 20 or the adapter. In some embodiments, the holes 72 may be aligned with the longitudinal axis 56, which may increase a length and volume of the holes 72.

In some embodiments, the neck portion 44 may include any suitable number of holes 72, such as, for example, between one and ten of the holes 72. In some embodiments, the neck portion 44 may include five of the holes 72. Similarly, in some embodiments, the neck portion 44 may include any suitable number of air vent channels 54, which may connect the holes 72. In some embodiments, the neck portion 44 may include one or more of: a first hole 72a, a second hole 72b, a third hole 72c, a fourth hole 72d, and a fifth hole 72e (which may be referred to in the present disclosure collectively as “holes 72”). In some embodiments, the neck portion 44 may include one or more of: a first air vent channel 54a, a second air vent channel 54b, a third air vent channel 54c, a fourth air vent channel 54d, and a fifth air vent channel 54e (which may be referred to in the present disclosure collectively as “air vent channels 54”).

In some embodiments, the air vent channels 54 may alternate between a first side of the neck portion 44 and a second side of the neck portion 44 opposite the first side, which may delay blood leaking through the plug 70. In some embodiments, the first hole 72a may be adjacent to the second hole 72b. In some embodiments, the second hole 72b may be adjacent to the third hole 72c. In some embodiments, the first air vent channel 54a may extend between the first hole 72a and the second hole 72b. In some embodiments, the second air vent channel 54b may extend between the second hole 72b and the third hole 72c. In some embodiments, the first air vent channel 54a may be disposed on an opposite side of the neck portion 44 as the second air vent channel 54b.

In some embodiments, the third hole 72c may be adjacent to the fourth hole 72d. In some embodiments, the third air vent channel 54c may extend between the third hole 72c and the fourth hole 72d. In some embodiments, the third air vent channel 54c may be disposed on the same side of the neck portion 44 as the first air vent channel 54a. In some embodiments, the fourth hole 72d may be adjacent the fifth hole 72e. In some embodiments, a fifth air vent channel 54e may extend from the flashback chamber 34 to the first hole 72a.

In some embodiments, the holes 72 may include a proximal-most hole, such as, for example, the fifth hole 70e. In some embodiments, the vent holes 42 may intersect the proximal-most hole. In some embodiments, the air pathway through the plug 70 may include the holes 72, the air vent channels 54, the vent holes 42, and the cavity 40. In some embodiments, the air vent channels 54 may be configured to allow air but not blood to pass. However, in other embodiments, blood may leak through the air vent channels 54 and through the air pathway, such that the air pathway is also a fluid pathway that includes blood. In some embodiments, the plug 70 may incorporate sudden contraction and sudden expansion, which may control and reduce blood flow and leakage. In some embodiments, the holes 72 may provide sudden expansion, while the air vent channels 54 may provide sudden contraction. In some embodiments, the holes 72 may hold a greater volume than the air vent channels 54, which may provide for the sudden expansion. In some embodiments, a proximal end 74 of the neck portion 44 and the distal end 46 of the body 38 may form the stepped surface 60, which may abut the needle hub 20 or the adapter.

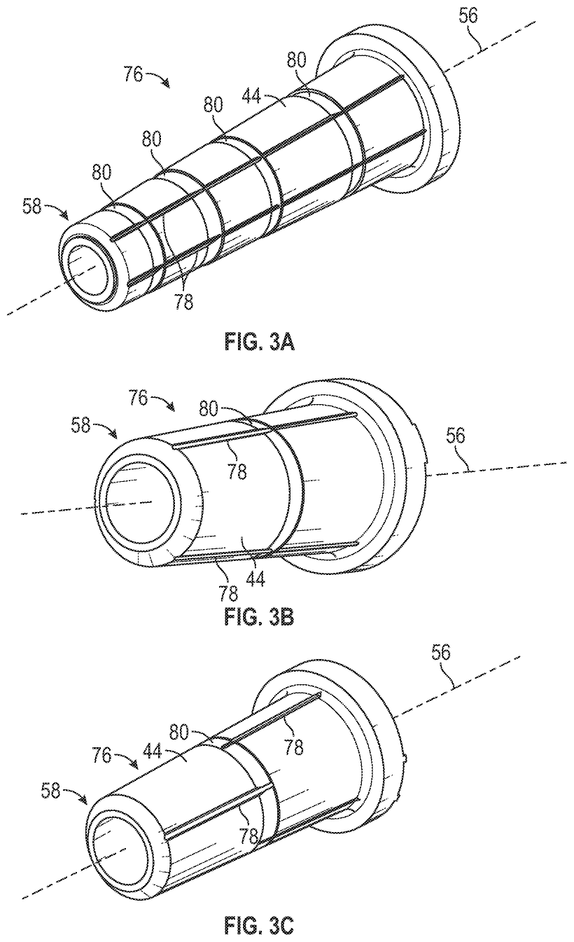

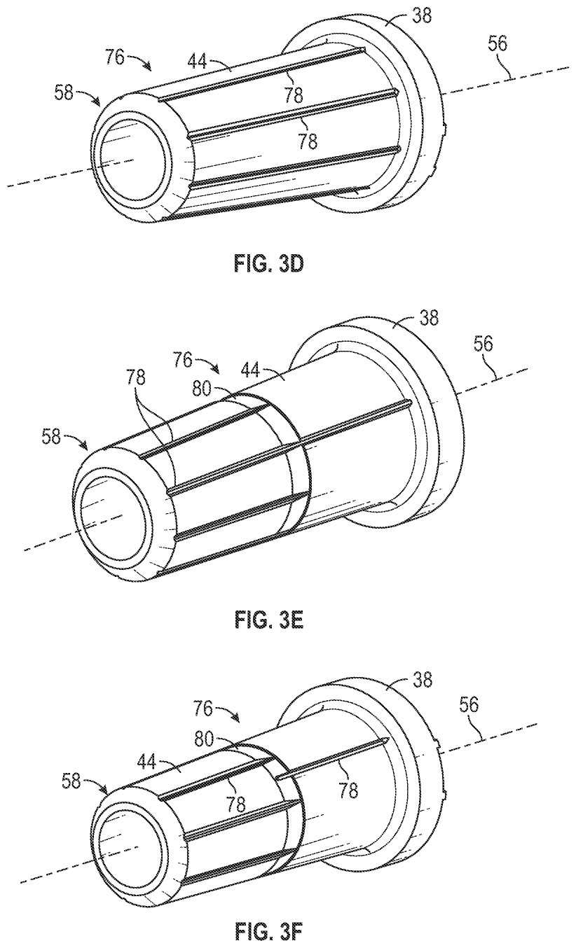

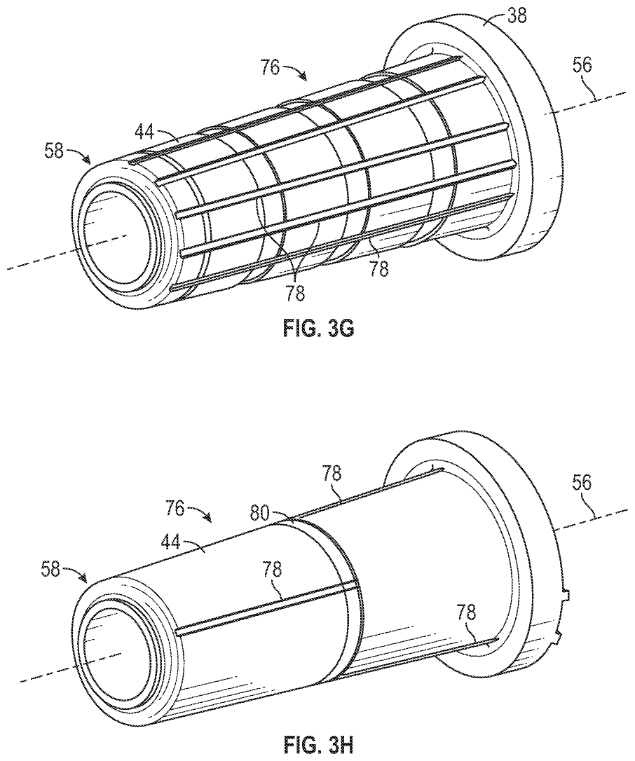

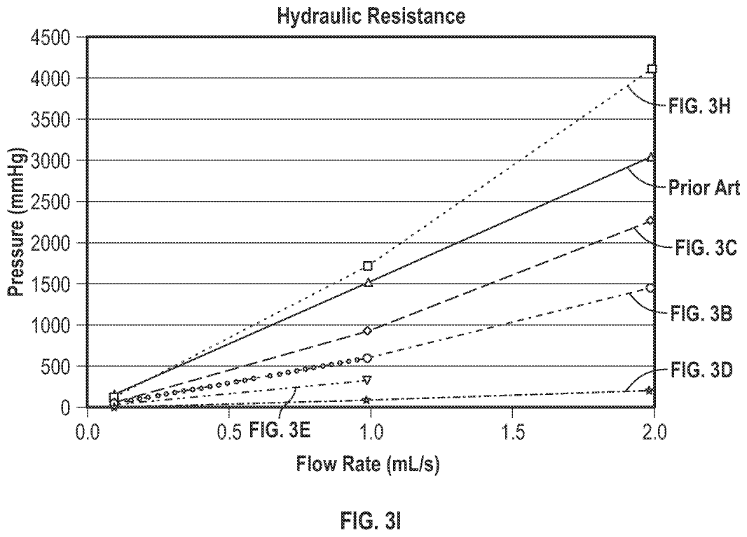

Referring now to FIGS. 3A-3G, a plug 76 is illustrated, according to some embodiments. In some embodiments, the plug 76 may be similar or identical to the plug 36 described with respect to FIGS. 1A-1H and/or the plug 70 described with respect to FIGS. 2A-2G in terms of one or more included components and/or operation.

In some embodiments, the neck portion 44 may include one or more longitudinal air vent channels 78, which may be generally aligned with the longitudinal axis 56 of the plug 76. In some embodiments, the neck portion 44 may include one or more transverse air vent channels 80 may connect the longitudinal air vent channels 78 to provide an air pathway from the distal end 58 of the neck portion 44. In some embodiments, the longitudinal air vent channels 78 and the transverse air vent channels 80 may pass air but not blood. In some embodiments, the longitudinal air vent channels 78 and the transverse air vent channels 80 may be disposed on an outer surface of the neck portion 44. In some embodiments, the longitudinal air vent channels 78 may be generally linear. In some embodiments, the transverse air vent channels 80 may be arc shaped.

In some embodiments, the longitudinal air vent channels 78 and/or the transverse vent air channels 80 may be arranged in various patterns, which may facilitate a pressure decrease compared to prior art plugs. As illustrated in FIG. 3A, in some embodiments, one or more of the transverse air vent channels 80 may be annular or semi-annular. In some embodiments the transverse air vent channels 80 may be shallower and/or wider than the longitudinal air vent channels 78. As illustrated in FIG. 3B, in some embodiments, the longitudinal air vent channels 78 may extend from the distal end 58 of the neck portion 44 along an entirety of a length of the neck portion 44. In some embodiments, the neck portion 44 may include two or more longitudinal air vent channels 78, which may be evenly spaced about the outer surface of the neck portion 44. For example, the neck portion 44 may include three longitudinal air vent channels 78, which may be evenly spaced around the neck portion 44 as in FIG. 1B, for example.

As illustrated in FIGS. 3C and 3F, in some embodiments, a first group of one or more of the longitudinal air vent channels 78 may extend from the distal end 58 of the neck portion 44 along a portion of the length of the neck portion 44. In some embodiments, the longitudinal air vent channels 78 of the first group may each include a distal end disposed at the distal end 58 of the neck portion 44 and a proximal end disposed at a particular transverse air vent channel 80. In some embodiments, a second group of one or more the longitudinal air vent channels 78 may each include a distal end at the particular transverse air vent channel 80 and a proximal end at or near the proximal end 74 of the neck portion 44. In some embodiments, the longitudinal air vent channels 78 of the first group and the longitudinal air vent channels 78 of the second group may alternate around the outer surface of the neck portion 44.

In some embodiments, a pattern of the longitudinal air vent channels 78 may be symmetric. As illustrated in FIG. 3C, in some embodiments, the first group of the longitudinal air vent channels 78 may include three longitudinal air vent channels 78 which may be evenly spaced around the neck portion 44. In some embodiments, the second group of the longitudinal air vent channels 78 may include three longitudinal air vent channels 78 which may be evenly spaced around the neck portion 44. As illustrated in FIG. 3H, in some embodiments, the first group of the longitudinal air vent channels 78 may include two longitudinal air vent channels 78 which may be evenly spaced around the neck portion 44. In some embodiments, the second group of the longitudinal air vent channels 78 may include two longitudinal air vent channels 78 which may be evenly spaced around the neck portion 44.

As illustrated in FIG. 3D, in some embodiments, the neck portion 44 may not include the transverse air vent channels 80. As illustrated in FIGS. 3D and 3G, in some embodiments, the neck portion 44 may include between one and twenty of the longitudinal air vent channels 78. For example, the neck portion 44 may include ten of the longitudinal air vent channels 78. In some embodiments, the neck portion 44 may include any suitable number of longitudinal air vent channels 78 and/or transverse air vent channels 80. As illustrated in FIG. 3E, in some embodiments, the neck portion 44 may include a combination of the longitudinal air vent channels 78 that extend along the entirety of the length of the neck portion 44 and the longitudinal air vent channels 78 that extend along a portion of the length of the neck portion 44.

Referring now to FIG. 3I, a graph of hydraulic resistance is illustrated, values of which are not limiting. Hydraulic pressure corresponding to use of embodiments of the plug 76 illustrated in FIGS. 3B-3E and 3H are shown in the graph, according to some embodiments. Prior art plugs may include a membrane to delay blood leakage. As illustrated in FIG. 3I, at a particular flow rate, embodiments of the plug 76, which utilize various channels within a neck portion instead of the membrane, may decrease pressure within a particular catheter assembly.

In some embodiments, unlike vent plugs known in the art, the plug 36 may not include a membrane to delay blood leakage due to the presence of the annular grooves 48 and the air vent channels 54. In some embodiments, the plug 36 may include a membrane in addition to the annular grooves 48 and/or the air vent channels 54

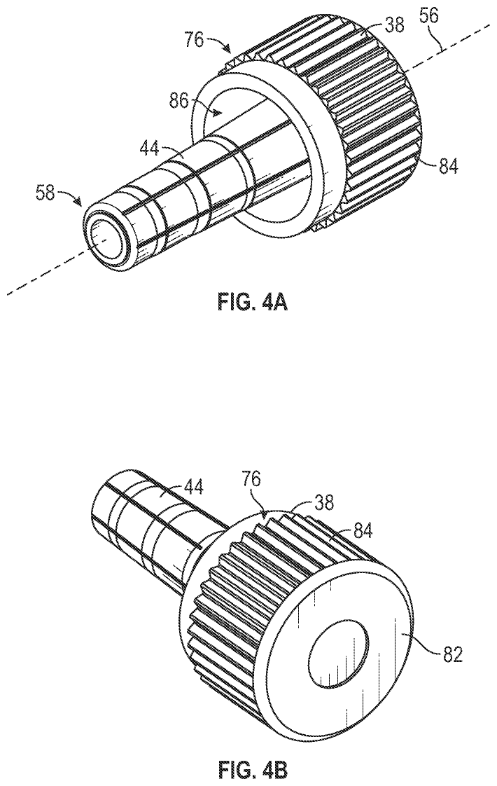

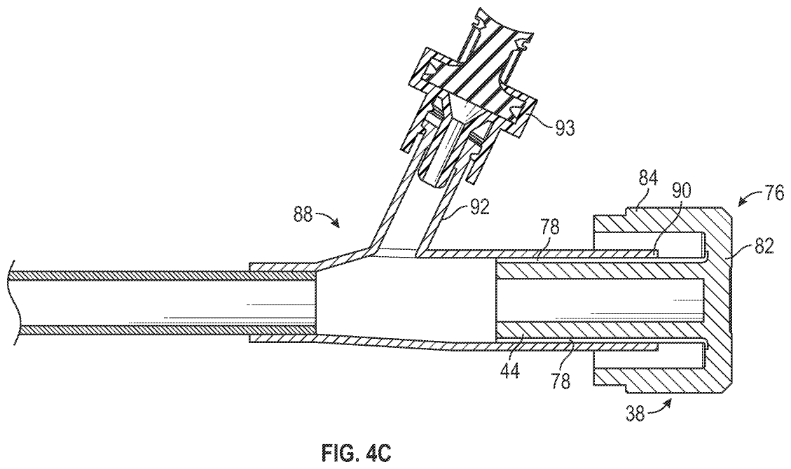

Referring now to FIGS. 4A-4C, in some embodiments, the plug 76 may include a Luer slip connector. In some embodiments, the body 38 may include an end wall 82 and a skirt 84 extending from the end wall 82 to define an annular receiving channel 86 between the skirt 84 and the neck portion 44. In some embodiments, an inner surface of the skirt 84 may be smooth. In some embodiments, the neck portion 44 may form a slip tip which may conform to dimensions of a needle hub (such as the needle hub 20 illustrated, for example, in FIG. 1A, 2A) or an adapter 88. In some embodiments, the slip tip and the needle hub or the adapter 88 may be pressed together and held by friction.

In some embodiments, the longitudinal air vent channels 68 may extend proximal to a distal end of the needle hub or a port 90 of the adapter 88. In some embodiments, the inner surface of the skirt 84 may be spaced apart from an outer surface of the needle hub 20 or the adapter 88 such that air may travel out of the longitudinal air vent channels 68 into the external environment. In some embodiments, the adapter 88 may include the port 90 and/or another port 92. In some embodiments, a needleless connector 93 or another suitable device may be coupled to the other port 92. In some embodiments, the adapter 88 may include a Y-adapter or another suitable adapter. In some embodiments, a medical device may be coupled to the Y-adapter for fluid administration or blood withdrawal. In some embodiments, an extension tube may extend from the adapter 88 to a side port of catheter assembly.

Referring now to FIGS. 5A-5C, in some embodiments, the plug 76 may include a Luer lock connector. In some embodiments, the inner surface of the skirt 84 may include threading 94. In some embodiments, the end wall 82 may include one or more vent holes 42 extending there through.

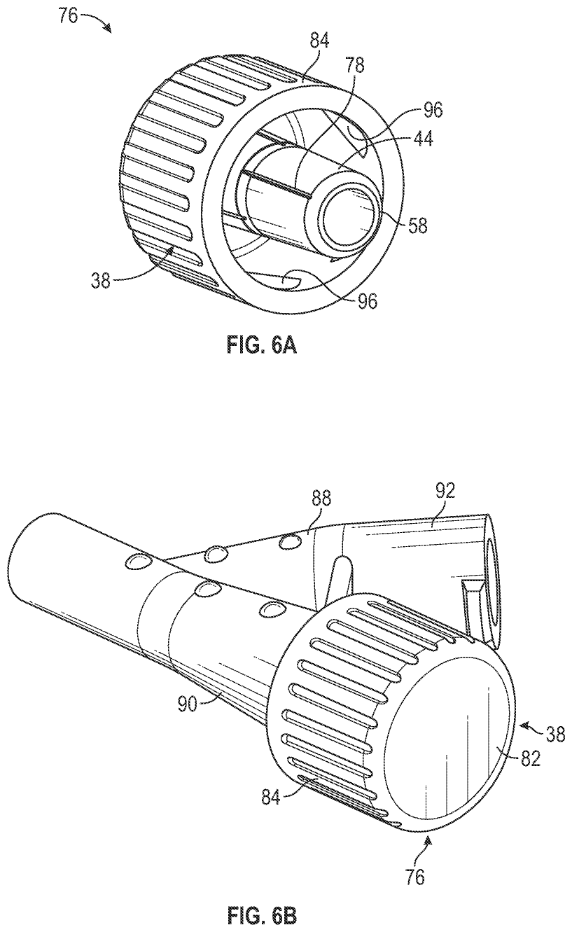

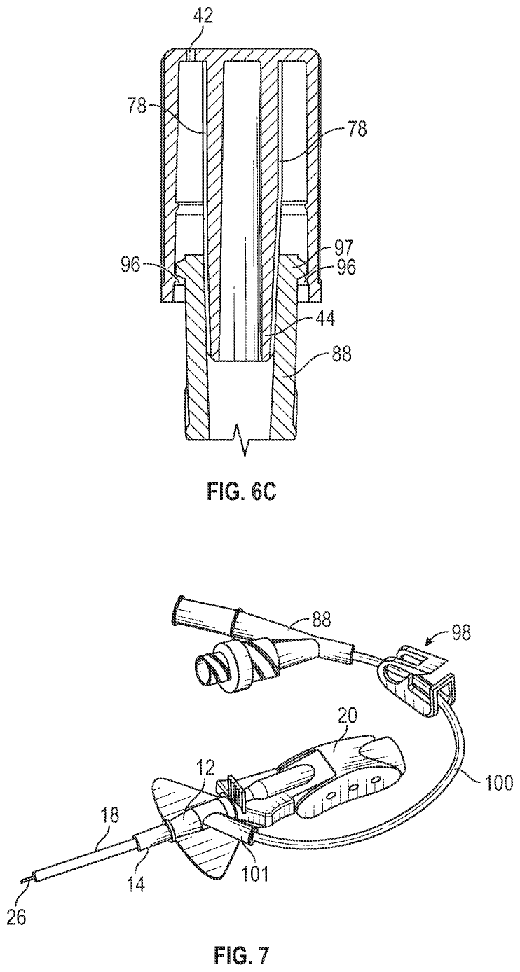

Referring now to FIGS. 6A-6C, in some embodiments, the inner surface of the skirt may include one or more snap features 96, which may include protrusions configured lock the plug 76 to the needle hub or the adapter 88 in response to movement of a flange 97 beyond the snap features 96. In some embodiments, the flange 97 may be annular. In some embodiments, the adapter 88 may include the flange 97 or the needle hub 20 (see, for example, FIG. 2C) may include the flange 97.

Referring now to FIG. 7, a catheter assembly 98 is illustrated, according to some embodiments. In some embodiments, the catheter assembly 98 may be similar or identical to the catheter assembly 10 described with respect to FIGS. 1A-2G in terms of one or more included components and/or operation. As illustrated in FIG. 7, the adapter 88 may be coupled to a proximal end of an extension tube 100, which may extend from a side port 101 of a catheter adapter 12.

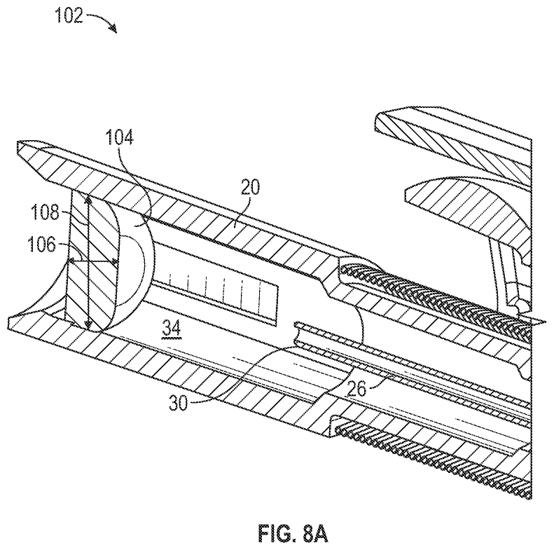

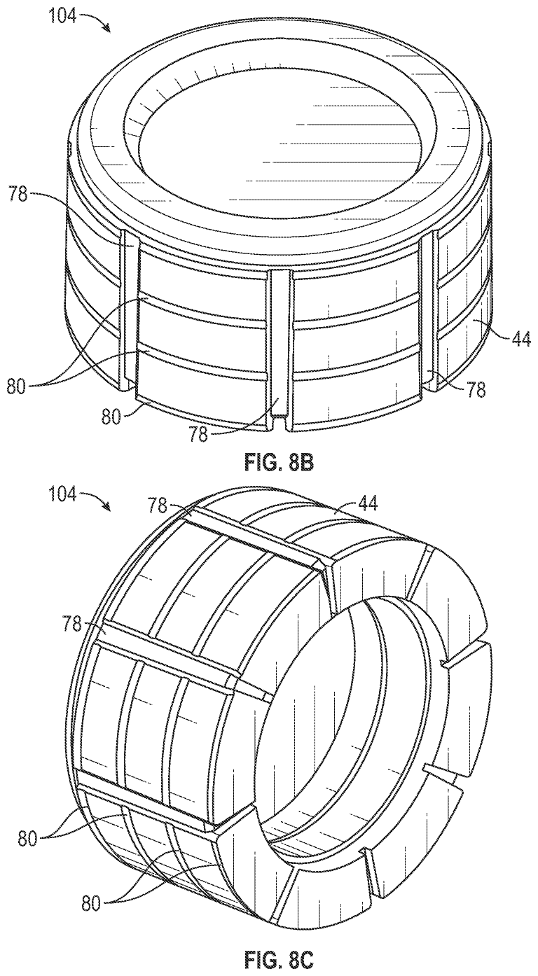

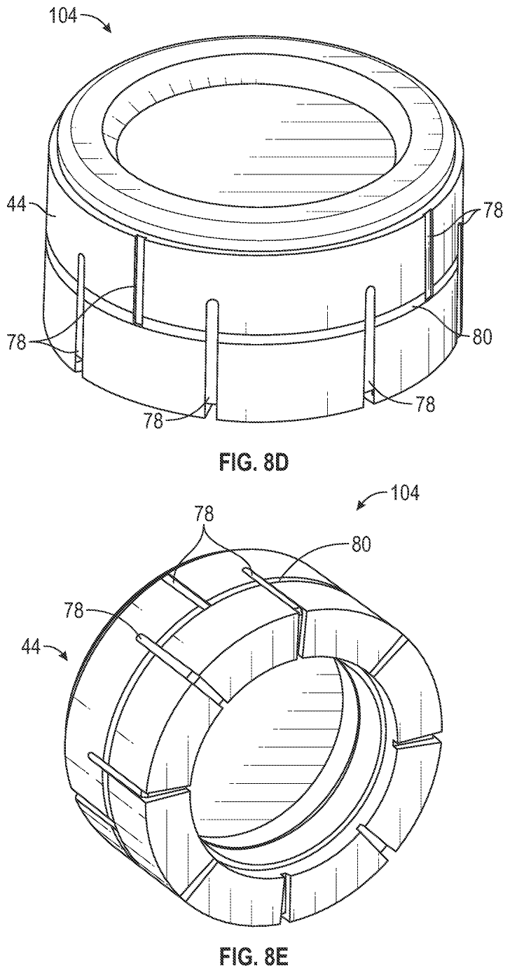

Referring now to FIGS. 8A-8E, a portion of a catheter system 102 is illustrated, according to some embodiments. In some embodiments, the catheter system 102 may be similar or identical to the catheter assembly 10 described with respect to FIGS. 1A-2G and/or the catheter assembly 98 described with respect to FIG. 7 in terms of one or more included components and/or operation. In some embodiments, the catheter system 102 may include a plug 104, which may be similar or identical in terms of one or more included components and/or operation to one or more of the following: the plug 36 described with respect to FIGS. 1A-1H, the plug 70 described with respect to FIGS. 2A-2G, and the plug 76 described with respect to FIGS. 3A-6C.

In some embodiments, the plug 104 may include the neck portion 44 but not the body 38. Thus, in some embodiments, the proximal end 74 of the neck portion 44 may correspond to a proximal end of the plug 104. In some embodiments, the plug 104 may be polyisoprene, polyimide, latex, polyurethane, nylon, polyethylene, plastic, or another suitable material. In some embodiments, the plug may include the longitudinal air vent channels 78 and/or the transverse air vent channels 80. In some embodiments, the longitudinal air vent channels 78 may be wider and/or deeper than the transverse air vent channels 80. In some embodiments, the plug 104 may be generally cylindrical. In some embodiments, a length 106 of the plug 104 in a distal-proximal direction may be less than a height 108 of the plug 104, and the plug 104 may reduce material and manufacturing costs.

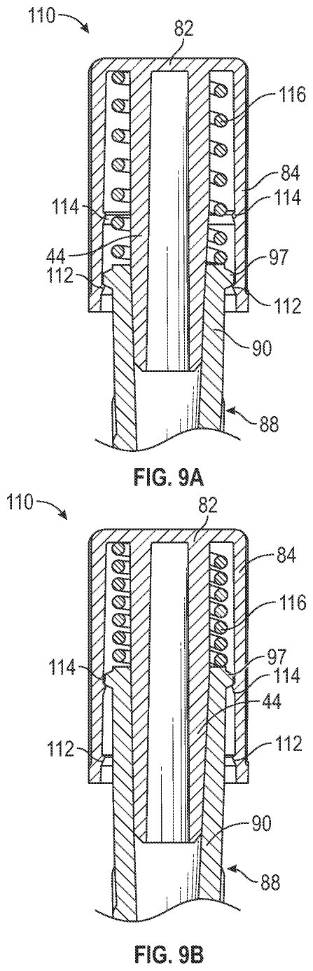

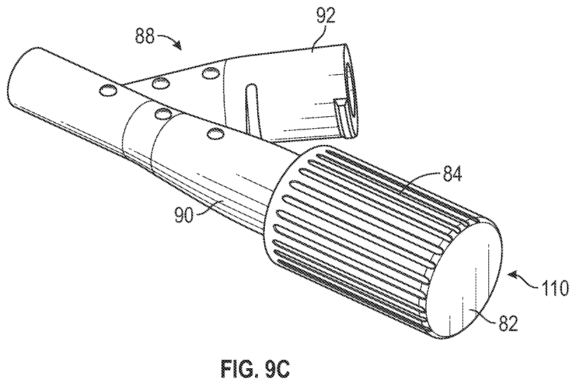

Referring now to FIGS. 9A-9C, a plug 110 is illustrated, according to some embodiments. In some embodiments, the plug 110 may be similar or identical in terms of one or more included components and/or operation to one or more of the following: the plug 36 described with respect to FIGS. 1A-1H, the plug 70 described with respect to FIGS. 2A-2G, the plug 76 described with respect to FIGS. 3A-6C, and the plug 104 described with respect to FIGS. 8A-8E. In some embodiments, the plug 110 may be used to plug the proximal end of a flashback chamber, such as, for example, the flashback chamber 34 illustrated in FIG. 1C.

In some embodiments, the inner surface of the skirt 84 may include one or more primary snap features 112, which may include protrusions configured lock the plug 110 to the needle hub or the adapter 88 in response to movement of the flange 97 proximally beyond the primary snap features 112. In some embodiments, the flange 97 and/or a particular primary snap feature 112 may be annular. In some embodiments, the adapter 88 may include the flange 97 or the needle hub 20 (see, for example, FIG. 2C) may include the flange 97.

In some embodiments, the inner surface of the skirt 84 may include one or more secondary snap features 114, which may include protrusions configured lock the plug 110 in a venting position in response to movement of the flange 97 proximally beyond the secondary snap features 114. In some embodiments, a particular secondary snap feature 114 may be annular. In some embodiments, in response to the plug 110 being in the venting position, the plug 110 may vent a catheter assembly. In some embodiments, a spring 116 may exert a force distally against the port 90 of the adapter 88, which may reduce a risk of the plug 110 actuating and moving to the venting position accidentally. In some embodiments, the user may actuate the plug 110 by moving the plug 110 to the venting position prior to inserting the catheter assembly into vasculature of the patient.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

We claim:

1. A plug for use in plugging a proximal end of a flashback chamber in an intravenous catheter assembly, the plug providing air venting of the flashback chamber, the plug comprising:
a body, comprising a cavity and a vent hole extending distally from the cavity;
a neck portion extending from a distal end of the body, wherein the neck portion comprises a plurality of annular grooves spaced apart by a plurality of disc-shaped portions, wherein a proximal-most disc-shaped portion of the plurality of disc-shaped portions is disposed proximate the body, wherein an outer edge of each of the plurality of disc-shaped portions comprises an air vent channel generally aligned with a longitudinal axis of the plug, wherein the air vent channel of the proximal-most disc-shaped portion is aligned with the vent hole.

2. The plug of claim 1, wherein a distal-most disc-shaped portion of the plurality of disc-shaped portions forms a distal end of the neck portion.

3. The plug of claim 1, wherein the proximal-most disc-shaped portion and the distal end of the body form a stepped surface.

4. The plug of claim 1, wherein a proximal end of the body comprises an opening of the cavity.

5. The plug of claim 1, wherein a surface of the cavity comprises a plurality of annular protrusions.

6. A plug for use in plugging a proximal end of a flashback chamber in an intravenous catheter assembly, the plug providing air venting of the flashback chamber, the plug comprising:
a body, comprising a cavity and a vent hole extending distally from the cavity;
a neck portion extending from a distal end of the body, wherein the neck portion comprises:
a plurality of holes extending through the neck portion generally perpendicular to a longitudinal axis of the plug;
a plurality of air vent channels disposed on an outer edge of the neck portion and connecting the plurality of holes, wherein an air pathway through the plug comprises the plurality of holes, the plurality of air vent channels, the vent hole, and the cavity.

7. The plug of claim 6, wherein the plurality of holes are spaced apart in a distal-proximal direction along the neck portion.

8. The plug of claim 7, wherein a first hole of the plurality of holes is adjacent to a second hole of the plurality of holes, wherein the second hole is adjacent to a third hole of the plurality of holes, wherein a first air vent channel extends between the first hole and the second hole, wherein a second air vent channel extends between the second hole and the third hole, wherein the first air vent channel is disposed on an opposite side of the neck portion as the second air vent channel.

9. The plug of claim 6, wherein the third hole is adjacent to a fourth hole of the plurality of holes, wherein a third air vent channel extends between the third hole and the fourth hole, wherein the third air vent channel is disposed on a same side of the neck portion as the first air vent channel.

10. The plug of claim 1, wherein a proximal end of the body comprises an opening of the cavity.

11. The plug of claim 6, wherein a surface of the cavity comprises a plurality of annular protrusions.

12. The plug of claim 6, wherein the plurality of holes comprises a proximal-most hole, wherein the vent hole intersects the proximal-most hole.

13. The plug of claim 1, wherein a proximal end of the neck portion and the distal end of the body form a stepped surface.

14. A plug for use in plugging a port of an adapter in an intravenous catheter assembly, the plug providing air venting of the port, the plug comprising:
a body;
a neck portion extending from the body,
a plurality of longitudinal air vent channels generally aligned with a longitudinal axis of the plug, a plurality of transverse air vent channels connecting the plurality of longitudinal air vent channels to provide an air pathway from a distal end of the neck portion.

15. The plug of claim 14, wherein the longitudinal air vent channels and the transverse longitudinal air vent channels pass air but not blood.

16. The plug of claim 14, wherein a proximal end of the neck portion and the distal end of the body form a stepped surface.

17. The plug of claim 14, wherein the body comprises an end wall and a skirt extending from the end wall to define an annular receiving channel between the skirt and the neck portion.

18. The plug of claim 17, wherein an inner surface of the skirt comprises threading.

19. The plug of claim 18, wherein the end wall comprises a vent hole extending there through.

20. The plug of claim 17, wherein an inner surface of the skirt comprises a plurality of snap features.
